(19) United States
(12) Patent Application Publication
(10) Pub. No.: US 2009/0036930 A1
(43) Pub. Date: Feb. 5, 2009

(54) Bone fixing device

(76) Inventor: David Mark Allison, Redcliffe (AU)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 10/585,473

(22) PCT Filed: Jan. 7, 2005
(86) PCT No.: PCT/AU05/00013

§ 371 Date: Jul. 7, 2006

(30) Foreign Application Priority Data
Jan. 8, 2004 (AU) 2004900432
Feb. 3, 2004 (AU) 2004900460

(51) Int. Cl. A61B 17/70

(52) U.S. Cl. 606/280; 606/70

(57) ABSTRACT

A bone fixing device (10) for fixing bone pieces together. The bone fixing device has a pair of spaced apart fixing portions (12), each having at least one hole (14) dimensioned such that a bone screw can extend there through; and a pair of substantially rigid connecting members (18) extending between the fixing portions, the connecting members being deformable without substantially losing rigidity. In use, each fixing portion is fixed to one or more of the bone pieces by a respective one of the bone screws such that the connecting members extend across one or more fissures between the bone pieces. The connecting members are simultaneously deformed such that the fissures are closed with the bone pieces held in compression.

### FIELD OF THE INVENTION

The present invention relates to bone fixing devices for fixing bone pieces together and also to a method of fixing bone pieces together.

### BACKGROUND

A common procedure in orthopaedic surgery is fixing bone pieces together. Often the bone pieces were originally one continuous bone piece which has separated either intentionally, for example in a re-alignment procedure, or unintentionally, as in the case of a fracture. Alternatively, it may be desirable to fix two or more bone pieces together such as when performing arthrodeses or joint fusions.

In almost all cases of such bone connections it is essential for the cut or fractured bone surfaces to be held together in close mutual confrontation. This intimacy of contact is usually referred to as compression. To form a strong bond between the bone pieces it is necessary for the bone surfaces to be held in well fitted contact during healing or union. In practice the simplest way of ensuring this contact is, where practical, to apply a compressive load to the bone portions in a direction substantially normal to the severed bone faces.

A known method to apply such a compressive loading is using bone screws. A first type of bone screw has a head attached to one end of a threaded shaft. The screw is screwed into the bone such that the head applies a compressive force directly to the bony cortex (or surface) of one of the pieces of bone to be held together.

A second type of bone screw comprises a shaft having two threaded portions separated by a shank devoid of thread. The two threaded portions are threaded in the same directions, but have a different pitch. Accordingly, as the screw is tightened a compressive load is applied to the bone faces.

One alternative to bone screws are bone plates. These are in various configurations, but are most commonly longer in one direction than in the other. A series of holes are provided in the plate in a single row along the longest direction. The plate is positioned across the fissure between the bone pieces such that longer dimension is generally orthogonal to the fissure. Bone screws with heads are screwed through the holes in the plate and into the bone. The plate holes can be elongated in the line of the longer dimension of the plate. If the screws are placed eccentrically away from the fissure, the bone surfaces will be drawn together on tightening.

In some such plates, the holes are provided with internal threads to engage complementary threads on the heads of the bone screws. Thus, the screws are locked to the plate.

In a further alternative, staples can be used to fix severed bone pieces together. Some staples can apply compression by virtue of their structure. Others can be tightened in various ways so as to bring about compression of the bone surfaces.

Each of the above mechanisms for fixing bone pieces together suffer from a number of deficiencies. For example, in practice it may not be possible to insert a screw in an appropriate direction due to the anatomical location of the pieces of bone to be fixed. Similarly, it may not be possible to use a bone plate due to size constraints and/or surgical access.

Staples can be difficult to use in that the bone can be split if the staple is too large. In addition, staples can remove themselves from the bone leaving an unstable fixation.

### SUMMARY OF THE INVENTION

According to a first aspect of the present invention, there is provided a bone fixing device for fixing bone pieces together, the bone fixing device comprising:
a pair of spaced-apart fixing portions, each having at least one hole dimensioned such that a fastener can extend therethrough; and,
a pair of substantially rigid connecting members extending between the fixing portions, the connecting members being deformable without substantially losing rigidity,
whereby, in use, each fixing portion is fixed to one or more of the bone pieces by the fasteners such that the connecting members extend across one or more fissures between the bone pieces, and the connecting members are simultaneously deformed such that the fissures are closed with the bone pieces held in compression.

According to a second aspect of the present invention, there is provided a modular system for fixing bone pieces together, the modular system comprising at least two bone fixing devices, each bone fixing device comprising:
a pair of spaced-apart fixing portions each having at least one hole dimensioned such that a fastener can extend therethrough; and,
a pair of substantially rigid connecting members extending between the fixing portions, the connecting members being deformable without losing rigidity,
whereby, in use, at least one of the fixing portions of each bone fixing device can be concentrically overlapped with a fixing portions of another bone fixing device such that a fastener can extend through the holes of the overlapped fixing portions.

According to a third aspect of the present invention, there is provided a method of fixing bone pieces separated by a fissure, comprising the steps of:
(a) providing at least one bone fixing device having a pair of spaced-apart fixing portions each having at least one hole being dimensioned such that a fastener can extend therethrough and a pair of deformable connecting members extending between the fixing portions;
(b) fixing one of the fixing portions to one bone piece using a fastener and the other fixing portion to another bone piece using another fastener such that the connecting members extend across the fissure; and,
(c) simultaneously deforming the connecting members such that the fixing portions are drawn together.

### BRIEF DESCRIPTION OF THE DRAWINGS

In order that the invention may be more easily understood, embodiments will now be described, by way of example only, with reference to the accompanying drawings, in which:

FIG. 1: is a plan view of a bone fixing device according to a first embodiment of the present invention;

FIG. 2: is a side view of the bone fixing device of FIG. 1;

FIG. 3: is a plan view of the bone fixing device of FIG. 1 showing a method of use of the device;

FIG. 4: is a plan view of the bone fixing device of FIG. 1 positioned across a fissure;

FIG. 5: is a side view of the bone fixing device of FIG. 4;

FIG. 6: is a plan view of the bone fixing device having been used to close the fissure of FIG. 4;

FIG. 7: is a side view of the bone fixing device of FIG. 6;

FIG. 8: is a plan view of two bone fixing devices according to a second embodiment of the present invention;

FIG. 9: is a side view of the bone fixing device according to the second embodiment;

FIG. 10: is a side view of the bone fixing device according to a third embodiment of the present invention;

FIG. 11: is a plan view of a bone fixing device according to a fourth embodiment of the present invention;

FIG. 12: is a plan view of a bone fixing device according to a fifth embodiment of the present invention;

FIG. 13: is a plan view of a bone fixing device according to a sixth embodiment of the present invention; and

FIG. 14: is a plan view of a bone fixing device according to a seventh embodiment of the present invention.

### DETAILED DESCRIPTION

FIGS. 1 to 7 show schematically a bone fixing device 10 according to a first embodiment of the present invention. The bone fixing device 10 has two fixing portions, in the form of annuli 12, each having a hole 14 for receiving a fastener, such as a bone screw 16. Two spaced apart substantially rigid connecting members 18 extend between the two annuli 12. In this embodiment the connecting members 18 are substantially parallel prior to deformation in use. The connecting members 18 join with the annuli 12 on the circumference of the annuli 12 so that the outer lateral dimensions of the connecting members are about the same as the outer diameter of the annuli 12 prior to deformation in use.

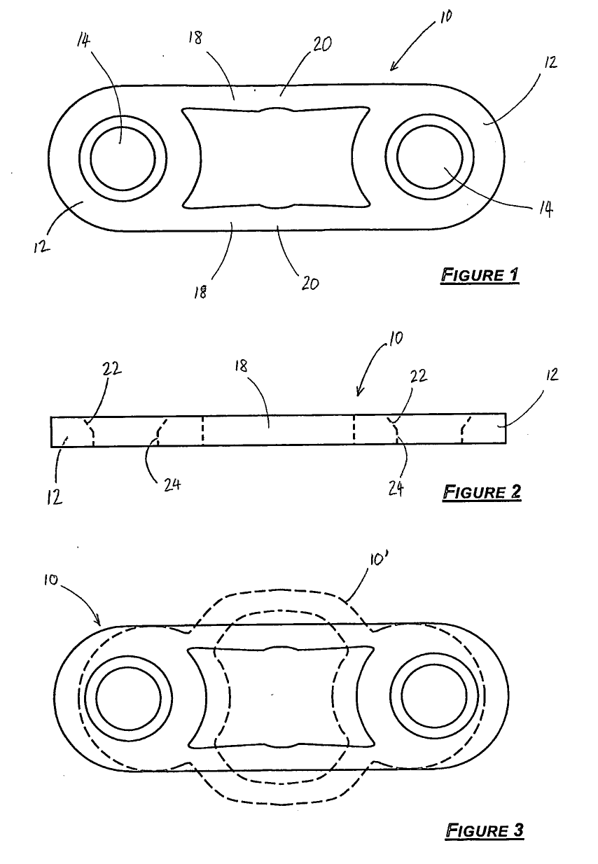

The connecting members 18 can be deformed, such as by using a pair of pliers, substantially without losing their rigidity, from the initial position (as shown in FIG. 1) such that the connecting members 18 are mutually separated. The separation will cause the annuli 12 to be drawn towards one another (as indicated by the broken lines 10′ in FIG. 3). The connecting members 18 will substantially maintain their position after deformation, and consequently the annuli 12 will also maintain their position closer together than prior to the deformation.

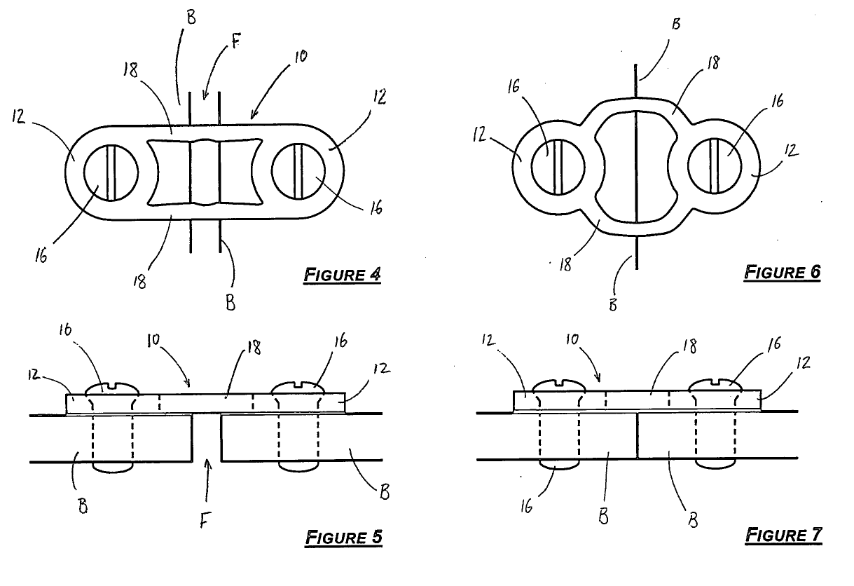

As shown in FIGS. 4 and 5, the bone fixing device 10 can be positioned across a fissure F between two bone pieces B. Each of the annuli 12 is attached to a respective one of the bone pieces B using a fastener in the form of a bone screw 16. Once the bone fixing device 10 is in place, the connecting members 18 can be deformed such that the annuli 12 are drawn together. As the connecting members 18 are deformed, the bone pieces B will also be drawn together closing the fissure F, as shown in FIGS. 6 and 7. Further deformation of the connecting members 18 will generate a compressive force on the bone pieces B at the contacting surfaces.

While the fissure F is shown as a single gap between the bone pieces B, it will be appreciated that there could be two or more fissures between the bone pieces B. For example, one or more bone pieces may be intercalated between the bone pieces B to which the fixing portions are attached.

As shown in FIG. 1, each of the connecting members 18 may be provided with a necked portion 20. Each of the necked portions 20 is a point of reduced resistance to deformation. The connecting members 18 will therefore bend most readily at the necked portions 20.

It will be appreciated that one or more neck portions 20 may be provided in each of the connecting members 18.

The holes 14 in each of the annuli 12 may have a frusto-conical portion 22, for receiving the head of a bone screw 16, and a cylindrical portion 24 for receiving part of the shaft of the bone screw 16.

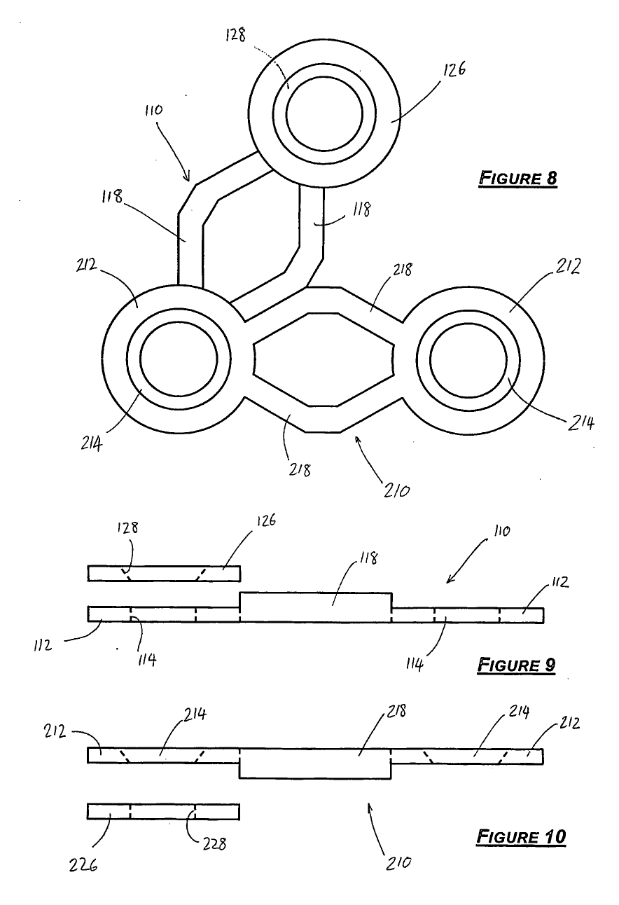

FIGS. 8 to 10 show two bone fixing devices 110 and 210 according to second and third embodiments respectively. In the second embodiment 110, shown in FIG. 9, two fixing portions, in the form of annuli 112, are provided between which two connecting members 118 extend.

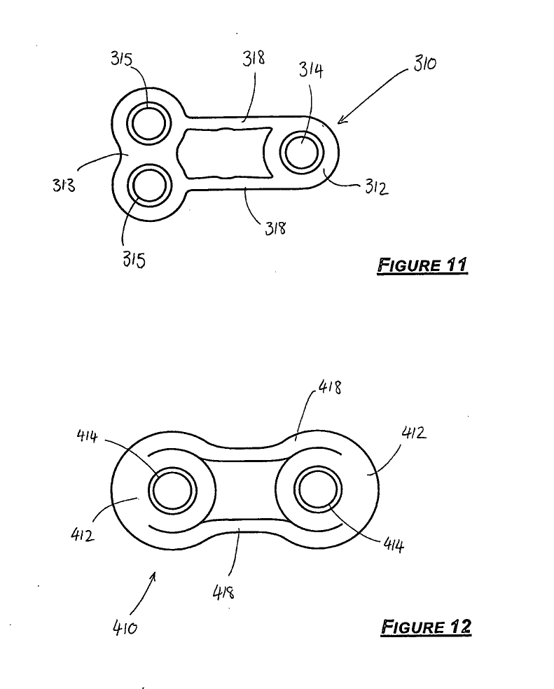

In the third embodiment 210, shown in FIG. 10, two fixing portions, in the form of annuli 212, are provided between which two connecting members 218 extend. As shown in FIG. 8, an annulus 212 of the bone fixing device 210 overlaps an annulus 112 of the bone fixing device 110 such that the holes 114, 214 of the respective annuli 112, 212 are concentric. Accordingly, the shaft of a bone screw (not shown) can extend through the holes 114, 214.

In this way, two or more bone fixing devices can be arranged to form a modular fixing system 150. The modular system 150 can be used to connect, and hold in compression, more than two bone pieces. Alternatively, the modular system 150 can be used to provide compression between two bone pieces which are separated by a nonlinear fissure. Alternatively or additionally, the modular system 150 may be used to provide at least bi-directional compression of bone pieces across a fissure.

In the modular fixing system 150, the overlapped annuli 112, 212 cooperate to receive and locate the bone screw which would fasten the bone fixing devices 110, 210 to the bone piece. The hole 214 in the annulus 212 is frusto-conical in shape, while the hole 114 in the annulus 112 is cylindrical in shape. Therefore, the hole 214 is dimensioned to receive the head of a bone screw and the hole 114 is dimensioned to receive the shaft of a bone screw.

The combined thickness of the annuli 112, 212 is approximately equal to the thickness of the annulus 12 of the bone fixing device 10. Thus, the thickness profile of the modular system 150 is substantially the same as that of the bone fixing device 10. It may be noted that the thickness of the annuli 112, 212 is approximately half the thickness of the respective connecting members 118, 218.

It will be apparent to the person skilled in the art that it may be necessary to provide a washer 124, 224 for the respective bone fixing devices 110, 210 such that an annulus 112, 212 in the modular system 150 which is not overlapped with another annulus can receive and locate a bone screw. Accordingly, each washer 126, 226 is provided with a hole 128, 228 which complements the annulus 112, 212 of the respective bone fixing device 110, 210.

While not illustrated in the figures, it will also be apparent that an alternative embodiment of bone fixing device could be provided having two annuli, one of the type illustrated in FIG. 9 and the other of the type illustrated in FIG. 10.

FIG. 11 shows schematically a bone fixing device 310 according to a fourth embodiment of the present invention. The bone fixing device 310 has two fixing portions. One fixing portion is in the form of an annulus 312 having a hole 314 for receiving a fastener such as a bone screw 16. The second fixing portion in the form of two conjoined annuli 313 such that there are two holes 315, each for receiving a fastener. Two connecting members 318 extend between the two fixing portions 312, 313, the connecting members 318 being deformable to draw the two fixing portions 312, 313 toward one another.

The bone fixing device 310 having a fixing portion in the form of the conjoined annuli 313 can be used to join three bone pieces together, for example, in an operation to fuse the scaphoid, trapezoid and trapezium bones in a patient's wrist. This operation is performed where arthritis in this joint has resulted in instability.

In such a fusion operation, the conjoined annuli 313 is fixed to both the trapezoid and trapezium bones by inserting bone screws 16 through each of the holes 315 and into a respective one of the trapezoid and trapezium bones. The annulus 312 is fixed to the scaphoid bone by inserting a bone screw 16 through the hole 314 and into the scaphoid bone. The connecting members 18 are then be simultaneously separated to draw the conjoined annuli 313 and the annulus 312 together. Compressive loads between both the scaphoid and trapezoid bones and the scaphoid and trapezium bones will be applied by the bone fixing device 310. Over time the trapezoid and trapezium bones will fuse with the scaphoid bone.

FIG. 12 shows schematically a bone fixing device 410 according to a fifth embodiment of the present invention. The bone fixing device 410 has two fixing portions, both generally in the form of an annulus 412 having a hole 414. Fasteners can be used to secure each of the annuli 412 to a bone piece.

Two connecting members 418 extend between the two annuli 412 such that each connecting member 418 is joined to each annulus 412 at a point remote from the opposing annulus 412. Accordingly, in their initial states, the annuli 412 of this bone fixing device 410 has a smaller separation when compared with the annuli 12 of the bone fixing device 10, when both bone fixing devices 10, 410 are provided with connecting members 18, 418 of the same length.

In use, the connecting members 418 can be simultaneously deformed such that the two annuli 412 are drawn together. Accordingly, a compressive load will be generated on the two bone pieces.

Clearly, the ease with which the connecting members can be deformed is at least partially dependent on the length of the connecting members. In addition, the maximum deflection of the fixing portions is also at least partially dependent on the length of the connecting members. The arrangement of the fixing portions 412 and the connecting members 418 allows the bone fixing device 410 to have a reduced overall length (and thus a reduced separation of the fixing portions 412) without shortening the length of the connecting members 418, when compared with the bone fixing device 10 of FIG. 1.

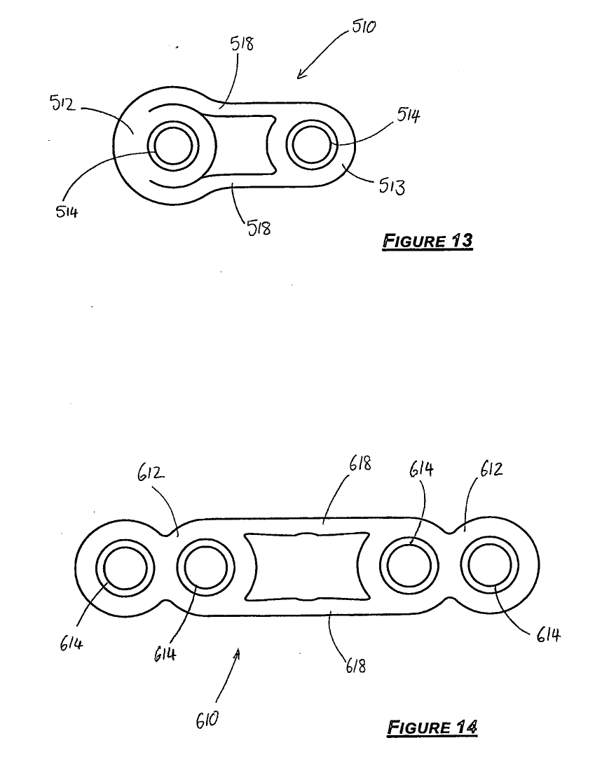

FIG. 13 shows schematically a bone fixing device 510 according to a sixth embodiment of the present invention. The bone fixing device 510 has two fixing portions 512, 513 both generally in the form of an annulus having a hole 514. Fasteners, such as bone screws 16, can be used to secure each of the fixing portion 512 to a bone piece.

Two connecting members 518 extend between the two fixing portions 512, 513. The first fixing portion 512 is joined to each connecting member 518 at a point remote from the second fixing portion 513. The second fixing portion 518 is joined to each connecting member 518 at a point near to the first fixing portion 512.

FIG. 14 shows schematically a bone fixing device 610 according to a seventh embodiment of the present invention. The bone fixing device 610 has two fixing portions, each in the form of two conjoined annuli 612 such that there are two holes 614 in each fixing portion. The holes 614 are able to receive a fastener such as a bone screw 16. Two connecting members 618 extend between the two fixing portions 612, the connecting members 618 being deformable to draw the two fixing portions 612 toward one another.

As shown in FIG. 14, the two holes 614 in each fixing portion are separated longitudinally with respect to the bone fixing device 610. In use, the load applied to each bone piece by the respective fixing portion is distributed between the two bone screws in each of the conjoined annuli 612. More than two holes could be incorporated at either or both ends of the device, either in line as shown in FIG. 14 or at an angle to the long axis of the device as shown in FIG. 11.

It will be understood to persons skilled in the art that modifications may be made without departing from the scope of the invention. For example, alternative embodiments of bone fixing devices may be conceived having combinations of the fixing portions described above in connection with the accompanying figures. In addition, alternative forms of fixing portions may be conceived which accommodate alternate forms of fastening means.

It will also be appreciated that, while the embodiments described above generate a compressive load on the respective bone pieces by separation of the connecting members, it is possible that a compressive load may be generated by pinching together the connecting members. It may also be possible to deform the connecting members in other ways providing that the fixing portions are drawn generally towards one another to generate a compressive load.

In the claims of this application and in the description of the invention, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

1. A bone fixing device for fixing bone pieces together, the bone fixing device comprising:
a pair of spaced-apart fixing portions, each having at least one hole dimensioned such that a fastener can extend therethrough; and,
a pair of substantially rigid connecting members extending between the fixing portions, the connecting members being deformable without substantially losing rigidity,
whereby, in use, each fixing portion is fixed to one or more of the bone pieces by the fasteners such that the connecting members extend across one or more fissures between the bone pieces, and the connecting members are simultaneously deformed such that the fissures are closed with the bone pieces held in compression.

2. A bone fixing device according to claim 1, wherein each fixing portion comprises an annulus.

3. A bone fixing device according to claim 1, wherein the connecting members are deformable symmetrically with respect to a line extending through the centre of the pair of fixing portions such that the fixing portions are drawn toward one another along the line.

4. A bone fixing device according to claim 3, wherein to achieve the symmetrical bending the connecting members are bent apart.

5. A bone fixing device according to claim 3, wherein to achieve the symmetrical bending the connecting members are pinched together.

6. A bone fixing device according to claim 1, wherein the connecting members are initially parallel.

7. A bone fixing device according to claim 1, wherein each connecting member has at least one point of weakness at which bending of the connecting member occurs in preference to elsewhere on the connecting member.

8. A bone fixing device according to claim 7, wherein each of the at least one point of weakness is a necked portion of the respective connecting member.

9. A bone fixing device according to claim 1, wherein at least one of the fixing portions comprises at least two holes each for receiving a fastener.

10. A modular system for fixing bone pieces together, the modular system comprising at least two bone fixing devices, each bone fixing device comprising:
a pair of spaced-apart fixing portions each having at least one hole dimensioned such that a fastener can extend therethrough; and,
a pair of substantially rigid connecting members extending between the fixing portions, the connecting members being deformable without losing rigidity,
whereby, in use, at least one of the fixing portions of each bone fixing device can be concentrically overlapped with a fixing portions of another bone fixing device such that a fastener can extend through the holes of the overlapped fixing portions.

11. A modular system according to claim 10, wherein the fixing portion is in the form of an annulus.

12. A modular system according to claim 11, further comprising at least one washer for overlapping with an annulus of a bone fixing device.

13. A modular system according to claim 12, wherein the hole within the at least one washer is frusto-conical in shape to receive the head of a bone screw.

14. A modular system according to claim 13, wherein the hole within the at least one washer is cylindrical in shape to receive the shaft of a bone screw.

15. A method of fixing bone pieces separated by a fissure, comprising the steps of:
(a) providing at least one bone fixing device having a pair of spaced-apart fixing portions each having at least one hole being dimensioned such that a fastener can extend therethrough and a pair of deformable connecting members extending between the fixing portions;
(b) fixing one of the fixing portions to one bone piece using a fastener and the other fixing portion to another bone piece using another fastener such that the connecting members extend across the fissure; and,
(c) simultaneously deforming the connecting members such that the fixing portions are drawn together.

16. A method according to claim 15, wherein the fixing portions each comprise an annulus, and step (a) involves overlapping an annulus of a first bone fixing device with an annulus of a second bone fixing device such that a fastening means can extend through both of a pair overlapped annuli.

17. A method according to claim 15, wherein simultaneously deforming the connecting members in step (c) further comprises substantially symmetrically deforming the connecting members.
